(19) United States
(12) Patent Application Publication
(10) Pub. No.: US 2026/0240818 A1
(43) Pub. Date: Aug. 20, 2026

(54) METHODS FOR THE TREATMENT OF FOOD ALLERGY

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); UNIVERSITÉ TOULOUSE III - PAUL SABATIER, Toulouse (FR)

(72) Inventors: Laurent REBER, Toulouse (FR); Jasper KAMPHUIS, Toulouse (FR)

(21) Appl. No.: 19/162,212

(22) PCT Filed: Mar. 7, 2024
(86) PCT No.: PCT/EP2024/056064

§ 371 Date: Sep. 4, 2025

(30) Foreign Application Priority Data
Mar. 8, 2023 (EP) 23305306.5

(51) Int. Cl. A61K 31/417; A61K 31/166; A61K 31/713; A61P 37/08

(52) U.S. Cl. CPC A61K 31/417; A61K 31/166; A61K 31/713; A61P 37/08

(57) ABSTRACT

The incidence of food allergy has dramatically increased over the last three decades in industrialized countries, now affecting more than 200 million people worldwide. Treatment options for food allergy are still limited: lifelong avoidance of the allergen is the main approach, followed by allergen-specific oral immunotherapy, which carries inherent risks. Food allergy occurs when type 2 immune responses are dysregulated, leading to production of allergen-specific IgE, and IgE-mediated mast cell degranulation upon re-exposure to the same allergen. Here, the Inventors demonstrate that dietary fructo-oligosaccharides, which are a major class of FODMAPs, can aggravate food allergic reactions through a mechanism involving formation of AGEs and activation of the receptor RAGE, implying that inhibition of the AGE/RAGE pathway represents a potential therapeutic strategy in food allergy.

### FIELD OF THE INVENTION

The present invention is in the field of medicine, in particular allergology.

### BACKGROUND OF THE INVENTION

The incidence of food allergy has dramatically increased over the last three decades in industrialized countries, now affecting more than 200 million people worldwide (Pawankar 2011; Sicherer and Sampson 2018; Turner and Campbell 2016). Treatment options for food allergy are still limited: lifelong avoidance of the allergen is the main approach, followed by allergen-specific oral immunotherapy, which carries inherent risks. Food allergy occurs when type 2 immune responses are dysregulated, leading to production of allergen-specific IgE, and IgE-mediated mast cell degranulation upon re-exposure to the same allergen.

The impact of dietary factors on the immune system and immune disorders is increasingly recognized over the last years. Regular diets contain poorly-absorbed fermentable carbohydrates, also known as FODMAPs (fermentable oligo-, di-, mono-saccharides, and polyols). While prebiotic dietary fibers such as cellulose and starch, and the resulting short chain fatty acids can have positive, anti-inflammatory, effects on (gut) health (Koh et al. 2016; Folkerts et al. 2018) and be protective against food allergy (Tan et al. 2016), unabsorbed but rapidly fermented carbohydrates like found in the FODMAP group could have negative health effects. Common FODMAPs include fructans, lactose, mannitol, sorbitol, and excess fructose (Staudacher and Whelan 2017). While restriction in dietary FODMAPs reduces symptoms of irritable bowel syndrome (IBS) and is routinely used in clinical practice (Whelan and Staudacher 2022), the effects of FODMAPs on the severity of food allergy remain unexplored.

The fermentation of FODMAPs by the gut microbiota can generate toxic glycating metabolites that favour the generation of Advanced End Glycation products (AGEs), which activate the pro-inflammatory Receptor for AGEs (RAGE). AGE formation can be prevented through treatment with pyridoxamine, an FDA-approved antiglycation agent normally used to prevent the progression of diabetic nephropathy (Williams et al. 2007). On the other end, several RAGE antagonists have been developed, among which azeliragon. Azeliragon has demonstrated a good safety profile in clinical trials in Alzheimer patients [NCT03980730]). Several publications indicate that RAGE plays an important role in mouse models of asthma (Oczypok et al. 2015; Ullah et al. 2014; Milutinovic et al. 2012). However, to date the effect of a blockade of AGEs formation and the effects of RAGE antagonists in food allergic responses have not been tested.

### SUMMARY OF THE INVENTION

The invention is defined by the claims. In particular, the present invention relates to a method of treating food allergy in a subject in need thereof comprising administering to said subject a therapeutically effective amount of a RAGE antagonist.

### DETAILED DESCRIPTION OF THE INVENTION

In the present invention, the Inventors demonstrate that dietary fructo-oligosaccharides, which are a major class of FODMAPs, can aggravate food allergic reactions through a mechanism involving formation of AGEs and activation of the receptor RAGE, implying that inhibition of the AGE/RAGE pathway represents a potential therapeutic strategy in food allergy.

Thus, in a first aspect, the present invention relates to a method of treating an IgE-mediated disease in a subject in need thereof comprising administering to said subject a RAGE antagonist.

As used herein, the term “IgE-mediated disease” refers to a disease characterized by increased levels of IgE. Typically, an IgE-mediated disease can be diagnosed by measuring the IgE levels in a biological sample (e.g., blood sample) from a subject suspected to suffer from an IgE-mediated disease, and/or by assessing at least one symptom associated with an allergic reaction including vomiting; diarrhea; abdominal cramps; hives; flushed skin or rash; tingling or itchy sensation in the mouth; face, tong or lip swelling; coughing; wheezing; dizziness; lightheadedness; swelling of the throat and vocal cords; hypotension; collapse. As example, IgE-mediated diseases include allergic diseases (e.g. food allergy, respiratory allergy, dermatitis), asthma, nasal polyposis, atopic keratoconjunctivitis, bullous pemphigoid, chronic urticaria, lymphoma (e.g. Sezary Syndrome, Hodgkin lymphoma), lupus, pancreatic cancer, nephrotic syndrome, nephritis, liver disease, cystic fibrosis, celiac disease, parasitosis, mucocutaneous candidiasis, Hyper IgE Syndrome (HIES), Kawasaki disease, Guillain-Barré syndrome and Kimura disease.

More particularly, the present invention relates to a method of treating food allergy in a subject in need thereof comprising administering to said subject a therapeutically effective amount of a RAGE antagonist. In some embodiments, the present invention also relates to a method for preventing food allergy in a subject at risk of being allergic to a food allergen, comprising administering to said subject a therapeutically effective amount of a RAGE antagonist.

As used herein, the term “allergy” or “allergic disease” refers to a reaction of immune system, particularly of specific IgE antibodies and mast cells. Typically, the IgE antibodies and antigen bind to the membrane receptors of mast cells and granulocytes and the antigen-antibody reaction releases inflammatory mediators leading to vasodilation and capillary permeability hyperactivity, causing tissue infiltration of inflammatory cells.

As used herein, the term “food allergy” refers to an immune system reaction occurring when a subject ingests a food allergen (i.e. a substance that causes or favor an allergic reaction when ingested). As example, food allergens include milk, celery, eggs, fish, crustacean shellfish, mollusks, mustard, tree nuts (e.g. almonds, hazelnuts, walnuts, Brazil nuts, cashews, pecans, pistachios, macadamia nuts), sesame, peanuts, lupin, wheat, gluten, soybeans, sulfur dioxide and sulphites. Symptoms associated with an allergic reaction includes symptoms related to gastrointestinal tract, cutaneous system, respiratory system or cardiovascular system. As example, symptoms associated with an allergic reaction includes vomiting; diarrhea; abdominal cramps; hives; flushed skin or rash; tingling or itchy sensation in the mouth; face, tong or lip swelling; coughing; wheezing; dizziness; lightheadedness; swelling of the throat and vocal cords; hypotension; collapse. Typically, a food allergy may be diagnosed with a blood test (e.g. to measure the allergy-related antibody IgE), an oral food challenge (e.g. ingesting the suspected allergen in gradually increasing doses) or an elimination diet (e.g. remove the suspected allergen from the diet of the subject for some weeks and observe the disappearance of symptoms).

As used herein, the term “subject” or “patient” denotes a mammal, such as a rodent, a feline, a canine, and a primate. Particularly, the subject according to the invention is a human. In some embodiments, the subject is allergic to an allergen. In some embodiments, the subject is at risk of being allergic to an allergen. As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal. In some embodiments, the allergen is a food allergen. In some embodiments, the food allergen is peanut. Thus, in some embodiments, the subject suffers from peanuts allergy. In some embodiments, the subject suffers from anaphylactic choc.

As used herein, the terms “treating”, “treatment” or “therapy” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The term encompasses reduction in the severity of symptoms due to food allergy. For example, a decrease in the severity of food allergy symptoms may be indicated by a decrease in the clinical score (see as Example Table 2). In some embodiments, the term “treatment” also refers to the preventive treatment of the food allergy in a subject at risk of being allergic to a food allergen. The treatment may be administered to a subject having a medical disorder or a subject likely to suffer from the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “efficient” denotes a state wherein the administration of one or more drugs to a subject permit to prevent, cure, delay the onset of, reduce the severity of, or ameliorate one or more symptoms of a disorder or recurring disorder, or to prolong the survival of a subject beyond that expected in the absence of such treatment. A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved.

### RAGE Antagonists

As used herein, the term “Advanced Glycation Endproducts” or “AGE” denotes a compound formed by irreversible nonenzymatic reactions between reducing sugars (e.g. glucose) and amino groups (e.g. in proteins, lipids or nucleic acids).

As used herein, the term “Receptor for Advanced Glycation Endproducts” or “RAGE” or “AGER” denotes a transmembrane protein encoded by AGER gene, that belongs to the immunoglobulin superfamily (Entrez Gene: 177; Ensembl: ENSG00000204305). The term includes the isoforms of the RAGE receptor, such as full-length RAGE, soluble RAGE (sRAGE) and dominant negative RAGE (dnRAGE). As example, RAGE ligands include AGE, S100p, S100A4, HMGB1, DIAPH1, quinolinic acid, amyloid-β and lipopolysaccharides. Enhanced levels of RAGE ligands have been reported in some disorders such as diabetes, Alzheimer's disease, rheumatoid arthritis and cancers.

As used herein, the term “RAGE antagonist” denotes a molecule that partially or fully blocks, inhibits or neutralizes a biological activity or expression of RAGE. As example, a RAGE antagonist partially or fully blocks, inhibits or neutralizes the interaction between RAGE and at least one of its ligands or inhibits the expression of AGER gene. In some embodiments, the RAGE antagonist directly binds to RAGE. In some embodiments, the RAGE antagonist directly interacts with at least one RAGE ligand. In order to identify a RAGE antagonist, a test may be necessary. Binding to RAGE and inhibition of the inhibition of the interaction between RAGE and at least one of its ligands may be determined by any competing assays well known in the art. For example, the assay may consist in determining the ability of the agent to be tested as RAGE antagonist to bind to RAGE. The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art.

Tests for determining the capacity of a compound to be a RAGE antagonist are well known to the person skilled in the art, as described in Dascalu et al. Chemistry. 2024. As example, protein-templated dynamic combinatorial chemistry (ptDCC) can be used to screen RAGE antagonist, as detailed in Dascalu et al. Chemistry. 2024. The functional assays may be envisaged such evaluating the ability to inhibit AGE2-BSA/sRAGE interaction.

In one embodiment, the antagonist according to the invention may be a low molecular weight compound, e.g. a small organic molecule (natural or not). The term “small organic molecule” refers to a molecule (natural or not) of a size comparable to those organic molecules generally used in pharmaceuticals. The term excludes biological macromolecules (e.g., proteins, nucleic acids, etc.). Preferred small organic molecules range in size up to about 10000 Da, more preferably up to 5000 Da, more preferably up to 2000 Da and most preferably up to about 1000 Da.

RAGE antagonists include but are not limited to azeliragon (CAS n°603148-36-3), TTP-4000, FPS-ZM1 (CAS n°945714-67-0), RP1, RAP (CAS n° 1092460-91-7), papaverine (CAS n° 61-25-6), carboxamides, aminoguanidine CAS n°5 79-17-4), pyridoxamine (CAS n° 85-87-0), pioglitazone (CAS n° 111025-46-8) and derivatives. As used herein, the term “derivative” refers to a compound derived from another after transformation of the latter. As example, the derivative may differ from the original compound by one or more atoms or functional groups. Small molecules that are RAGE antagonists are well known in the art and includes as example those described in the international patent applications WO02/070473, WO03/075921, WO2007/089616, WO2008/153957.

In some embodiments, the RAGE antagonist is azeliragon. As used herein, the term “Azeliragon” or “TTP488” or “PF-04494700” refers to 3-[4-[2-butyl-1-[4-(4-chlorophenoxy)phenyl]imidazol-4-yl]phenoxy]-N,N-diethylpropan-1-amine. An exemplary representation of the chemical structure of azeliragon is shown below:

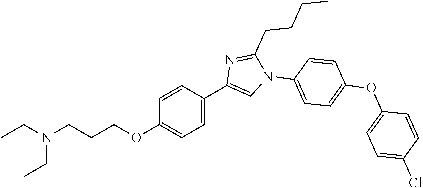 [SMILES: CCCCc1nc(-c2ccc(OCCCN(CC)CC)cc2)cn1-c1ccc(Oc2ccc(Cl)cc2)cc1]

As used herein, the term “FPS-ZM1” refers to 4-Chloro-N-cyclohexyl-N-(phenylmethyl)benzamide. An exemplary representation of the chemical structure of FPS-ZM1 is shown below:

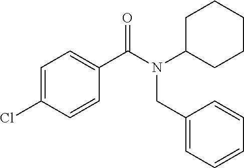 [SMILES: O=C(c1ccc(Cl)cc1)N(Cc1ccccc1)C1CCCCC1]

In some embodiments, the present invention relates to a RAGE antagonist for use in the treatment or prevention of food allergy, wherein an effective amount of said RAGE antagonist is administered to a subject suffering from food allergy or to a subject at risk of being allergic to a food allergen; and wherein said RAGE antagonist is Azeliragon or FPS-ZM1.

In one embodiment, the antagonist according to the invention (i.e. RAGE antagonist) is an antibody. Antibodies directed against RAGE can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against RAGE can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-RAGE single chain antibodies. Compounds useful in practicing the present invention also include anti-RAGE antibody fragments including but not limited to F(ab′) 2 fragments, which can be generated by pepsin digestion of an intact antibody molecule, and Fab fragments, which can be generated by reducing the disulfide bridges of the F(ab′) 2 fragments. Alternatively, Fab and/or scFv expression libraries can be constructed to allow rapid identification of fragments having the desired specificity to RAGE. Humanized anti-RAGE antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

In another embodiment, the antibody according to the invention is a single domain antibody directed against RAGE. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH. The VHH according to the invention can readily be prepared by an ordinarily skilled artisan using routine experimentation. The VHH variants and modified form thereof may be produced under any known technique in the art such as in-vitro maturation. VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then, for this invention, neutralizing aptamers of RAGE are selected.

In one embodiment, the compound according to the invention is a polypeptide. In a particular embodiment the polypeptide is an antagonist of RAGE and is capable to prevent the function of RAGE. Particularly, the polypeptide can be a mutated ligand of RAGE, a mutated RAGE protein, a truncated RAGE protein or a similar protein without the function of RAGE. In some embodiments, the RAGE antagonist is a fusion protein. As example, the fusion protein may comprise a RAGE polypeptide (e.g. RAGE binding site) linked to a second non RAGE polypeptide. In one embodiment, the polypeptide of the invention may be linked to a cell-penetrating peptide to allow the penetration of the polypeptide in the cell. The term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012). The polypeptides of the invention may be produced by any suitable means, as will be apparent to those of skill in the art. In order to produce sufficient amounts of polypeptide or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known. When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli. In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications. Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa). In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the RAGE antagonist according to the invention inhibits RAGE gene expression.

Small inhibitory RNAs (siRNAs) can also function as inhibitors of RAGE expression for use in the present invention. RAGE gene expression can be reduced by contacting a subject or cell with a small double stranded RNA (dsRNA), or a vector or construct causing the production of a small double stranded RNA, such that RAGE gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, MT. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). In some embodiments, the RAGE antagonist is a siRNA. In some embodiments, the RAGE antagonist is a siRNA directed against RAGE. In some embodiments, the RAGE antagonist is ARO-RAGE (Arrowhead Pharmaceuticals). In some embodiments, the RAGE antagonist is ADS-015 (CAS n°: 2756997-71-2). In some embodiments, the RAGE antagonist is AC000292.

Ribozymes can also function as inhibitors of RAGE gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of RAGE mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays. Both antisense oligonucleotides and ribozymes useful as inhibitors of RAGE gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing RAGE. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles are provided in Kriegler, 1990 and in Murry, 1991. Preferred viruses for certain applications are the adenoviruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter can also be, e.g., a viral promoter, such as CMV promoter or any synthetic promoters.

### Pharmaceutical Compositions

In another aspect, the present invention relates to a pharmaceutical composition comprising a RAGE antagonist (e.g. Azeliragon, FPS-ZM1 or ARO-RAGE) for use in the treatment of food allergy. In some embodiments, the present invention relates to a pharmaceutical composition comprising a RAGE antagonist and at least one further therapeutic agent. In some embodiments, the present invention relates to a pharmaceutical composition comprising a RAGE antagonist, at least one further therapeutic agent selected from the list comprising or consisting of antihistamines (e.g. cetirizine, fexofenadine, levocetirizine, loratadine, brompheniramine, chlorpheniramine, clemastine, diphenhydramine, ketotifen, naphazoline, pheniramine), immunotherapies (e.g. Grastek, Oralair, Ragwitek, Odactra, AR101, AR201, Viaskin, Omalizumab), decongestants (e.g. pseudoephedrine, phenylephrine, omymetazoline), corticosteroids, steroids (e.g. beclomethasone, ciclesonide, fluticasone furoate, mometasone, budesonide, fluticasone, triamcinolone, dexamethasone, loteprednol, prednisone), mast cell stabilizers (e.g. cromolyn sodium, lodoxamide-tromethamine, nedocromil, pemirolast), leukotriene modifiers (e.g. motelukast), adrenalin; and optionally pharmaceutically acceptable excipients. In some embodiments, the at least one further therapeutic agent is selected from the list consisting of cetirizine, fexofenadine, levocetirizine, loratadine, brompheniramine, chlorpheniramine, clemastine, diphenhydramine, ketotifen, naphazoline, pheniramine, grastek, oralair, ragwitek, odactra, AR101, AR201, Viaskin, Omalizumab, pseudoephedrine, phenylephrine, omymetazoline, beclomethasone, ciclesonide, fluticasone furoate, mometasone, budesonide, fluticasone, triamcinolone, dexamethasone, loteprednol, prednisone, cromolyn sodium, lodoxamide-tromethamine, nedocromil, pemirolast, motelukas, adrenalin.

Any therapeutic agent of the invention may be combined with pharmaceutically acceptable excipients, and optionally sustained-release matrices, such as biodegradable polymers, to form therapeutic compositions. “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The form of the pharmaceutical compositions, the route of administration, the dosage and the regimen naturally depend upon the condition to be treated, the severity of the illness, the age, weight, and sex of the patient, etc. The pharmaceutical compositions of the invention can be formulated for a topical, parenteral, intranasal, intraocular, intravenous, intramuscular or subcutaneous administration and the like. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The doses used for the administration can be adapted as a function of various parameters, and in particular as a function of the mode of administration used, of the relevant pathology, or alternatively of the desired duration of treatment. In addition, other pharmaceutically acceptable forms include, e.g. tablets or other solids for oral administration; time release capsules; and any other form currently can be used.

The invention will be further illustrated by the following figures and examples. However, these examples and figures should not be interpreted in any way as limiting the scope of the present invention.

### FIGURES

FIG. 1. Fructo-oligosaccharides (FOS) can amplify peanut allergic responses in a mouse model. (A) Protocol outline. Mice are maintained on control or FOS-enriched diets throughout the experiment. After 3 weeks, mice are sensitized with peanut extract (PE) and the adjuvant alum. Mice are then challenged orally with PE daily for one week and sacrificed 24 h after the last PE challenge. (B-D) average clinical score of days 1-3 (B), incidence of diarrhoea (C) and levels of peanut-specific IgE (D). Data are pooled from 2 independent experiments with a total of 9-10 mice per group. * or **: P<0.05 or 0.01 vs indicated group.

FIG. 2. Blocking the formation Advanced Glycation End produces (AGEs) by treatment with pyridoxamine eliminates the allergy-aggravating effects of the high-FOS diet. (A) Protocol outline. Mice on a control or FOS-enriched diet received anti-glycation agent pyridoxamine in drinking water at 1 mg/ml or water only (as a control) throughout the experiment. 3 weeks after beginning of the treatment, mice are sensitized with peanut extract (PE) and the adjuvant alum. Mice are then challenged orally with PE daily for one week and sacrificed 24 h after the last PE challenge. (B-D) average clinical score of days 1-3 (B), incidence of diarrhoea (C), and peanut specific IgE (D). Clinical score and diarrhoea data represent independent experiments with a total of 8-10 mice per group. ELISA data represents 1 experiment with 4-5 mice per group. *, *, ***: P<0.05, 0.01, 0.001 vs indicated group; ns: not significant

FIG. 3. Blocking the receptor of Advanced Glycation End Products (RAGE) with FPS-ZM1 eliminates the allergy-aggravating effects of the high-FOS diet. (A) Protocol outline. Mice on a control or FOS-enriched diet were treated twice-weekly intraperitoneally (i.p.) with the RAGE antagonist FPS-ZM1 (0.5 or 1 mg/kg b.w.) or vehicle as a control. 3 weeks after beginning of the treatment, mice are sensitized with peanut extract (PE) and the adjuvant alum. Mice are then challenged orally with PE daily for one week and sacrificed 24 h after the last PE challenge. (B-D) average clinical score of days 1-3 (B), incidence of diarrhoea (C), and peanut specific IgE (D). Clinical score and diarrhoea data represent independent experiments with a total of 8-10 mice per group. ELISA data represents 1 experiment with 4-5 mice per group. *, **, ***: P<0.05, 0.01, 0.001 vs indicated group; ns: not significant

FIG. 4. Blocking the receptor of Advanced Glycation End Products (RAGE) with oral treatment with azeliragon eliminates the allergy-aggravating effects of the high-FOS diet. (A) Protocol outline. Mice on a control or FOS-enriched diet were treated twice-weekly by oral gavages with the RAGE antagonist azeliragon (1 mg/kg b.w.) or vehicle as a control. 3 weeks after beginning of the treatment, mice are sensitized with peanut extract (PE) and the adjuvant alum. Mice are then challenged orally with PE daily for one week and sacrificed 24 h after the last PE challenge. Mice (B-D) average clinical score of days 1-3 (B), incidence of diarrhoea (C), and peanut specific IgE (D). Data represents 1 experiment with 4-5 mice per group.

FIG. 5. Mice genetically deficient for the receptor of Advanced Glycation End Products (RAGE) are protected from food allergy. (A) Protocol outline. Mice are maintained on control or FOS-enriched diets throughout the experiment. After 3 weeks, mice are sensitized with peanut extract (PE) and the adjuvant alum. Mice are then challenged orally with PE daily for one week and sacrificed 24 h after the last PE challenge. Mice (B-D) average clinical score of days 1-3 (B), incidence of diarrhoea (C), and peanut specific IgE (D). Data represents 2 experiments with a total of 7-8 mice per group.

### EXAMPLE

### Material and Methods

### Peanut Allergy Model

All mice were bred and maintained on a peanut- and soy-free diet under specific pathogen-free conditions until the start of the experiment. For the experiment using mice genetically deficient for RAGE, Agertm2.1 (cre/ERT2) Blh/2J mice were bought from Jackson Labs and bred in the local animal facility. At 5 weeks of age mice are put on custom experimental diets (Ssniff Spezialdiäten, Germany), diet compositions are specified in Table 1.

On days 1, 7, and 21 mice were sensitized with 500 μg of peanut extract (PE) together with 2 mg of Imject alum (Thermo Fisher, France) by intraperitoneal (IP) injection (100 μl in PBS). Starting from day 35, for 7 days, mice were challenged with 30 mg of PE in NaHCO₃ (0.1M) by oral gavage, and clinical score (Table 2) and occurrence of diarrhoea was registered for 1 hours after each gavage. After the last challenge, blood is collected for serum extraction. Non-allergic control animals were sham sensitized (2 mg alum but no peanut extract) but challenged like the other experimental groups.

Pyridoxamine: pyridoxamine dihydrochloride (Sigma-Aldrich, France) is added to drinking water at 1 mg/ml throughout the experiment.

RAGE antagonist FPS-ZM1: twice weekly 100 μl IP injections of FPS-ZM1 in PBS (Sigma Aldrich, France) at 0 (control group), 0.5, or 1 mg/kg b.w. were performed throughout the experiment.

Azeliragon: Azeliragon (Medchemexpress, France), was administered twice weekly at 0 or 1 mg/kg b.w. by oral gavage in PBS.

Clinical Scoring after Allergen Challenge

Clinical score (Table 2) was continually observed from 15 to 50 minutes after oral allergen challenge and noted for each mouse. For analysis purposes, the average clinical score of the first 3 days of challenge is calculated for each mouse.

### Peanut-Specific IgE ELISA

Peanut Extract (PE) was coated on ELISA plates at 5 μg/ml in 0.05 M sodium carbonate-bicarbonate buffer (Sigma-Aldrich, France) over-night at 4° C., plates were washed 3 times with PBS-0.05% Tween 20 (Sigma-Aldrich, France). Apart from the coating antibody, which was replaced by PE as described before, Mouse IgE ELISA kit (Invitrogen 88-50460-88; Thermo Fisher, France) was used according to manufacturer's instructions. Sample preparation: Mouse serum from blood collected 1 hour after the last oral peanut challenge was diluted 1/50 in ELISA assay buffer (1×) from the ELISA kit used. The colorimetric reaction was stopped by addition of H2SO4 (5M) and absorbance was recorded at 450 nm.

### Peanut Extract (PE) Preparation

Peanut extract was prepared from commercially available partially defatted raw peanut flour (Bio Planete). Peanut flour was suspended in 0.1M NaHCO₃ at 10% (w/v), the suspension was brought to pH 10 by titration with NaOH (1M) and proteins were brought in solution under constant agitation at 4° C. for 8 hours. Supernatant containing the protein fraction was recovered by centrifugation for 60 min at 4,500 g (4° C.). The protein solution was brought to pH 4 by titration under constant agitation to precipitate the proteins. The protein suspension was centrifuged for 25 min at 4,500 g (4° C.) and supernatant discarded. The protein fraction is resolubilized in 0.1M NaHCO3 at pH8 to obtain a final concentration of 120 mg/ml protein in solution.

### Statistics

Experimental groups were compared by One-Way ANOVA, selected groups were compared using Holm-Sidak's correction for multiple comparisons. For diarrhoea data, pairwise comparisons of survival curves were performed and analysed by Mantel-Cox log-rank test.

### Results

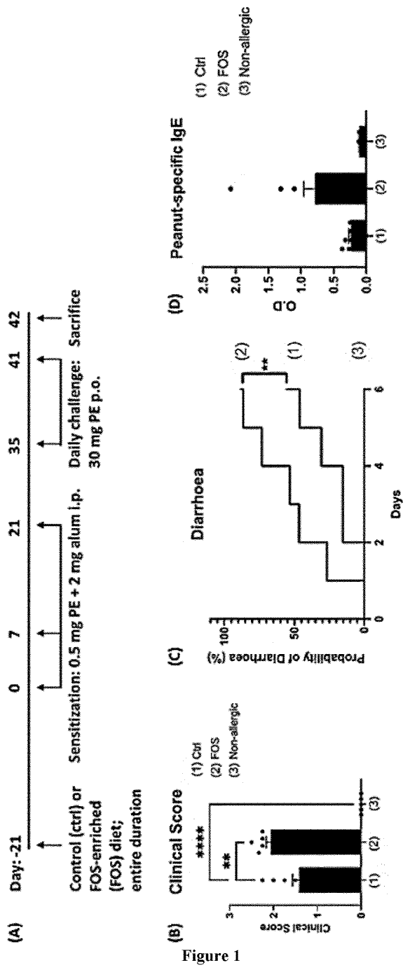

A high fructo-oligosaccharide (FOS) diet aggravates food allergy in a mouse model of peanut allergy (presented in FIG. 1A), indicated by an increased clinical score (FIG. 1B), increased incidence of diarrhoea upon allergen exposure (FIG. 1C), and increases in serum peanut-specific IgE levels (FIG. 1D).

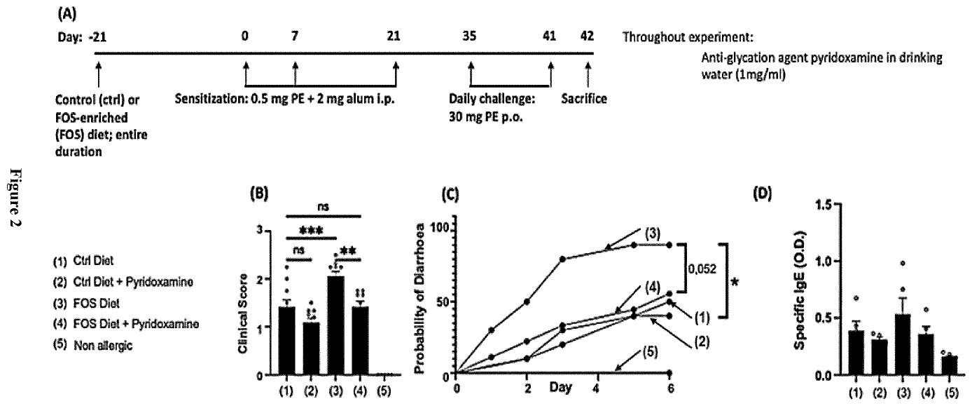

Dietary fructo-oligosaccharides (FOS) induce formation of advanced glycation end products (AGEs), mediated by the microbiota (presented in FIG. 2A). Our data in FIG. 2 indicate that treating peanut sensitized mice with the anti-glycation agent pyridoxamine in drinking water can block the aggravating effects of the fructo-oligosaccharides (FOS)-enriched diet on peanut allergic reactions in mice. Specifically, pyridoxamine treatment reduced both the clinical score (FIG. 2B), incidence of diarrhoea (FIG. 2C) and serum peanut-specific IgE levels (FIG. 2D) in this peanut allergy model. Altogether, these data demonstrate that increased glycation reactions induced by dietary fermentable carbohydrates can aggravate peanut allergic reactions.

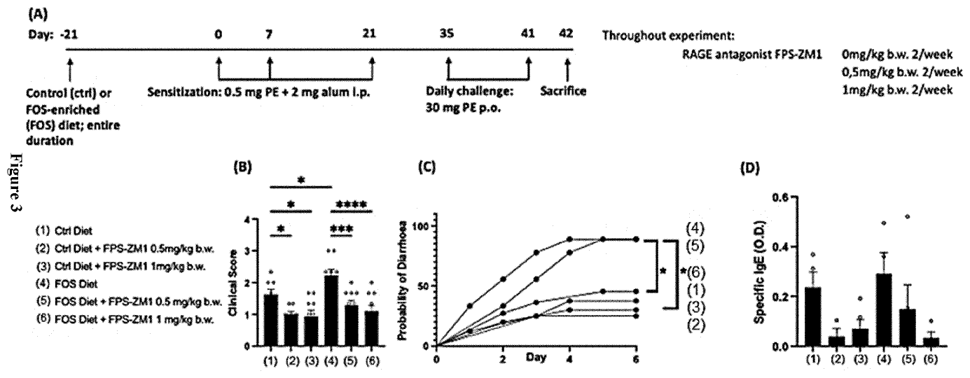

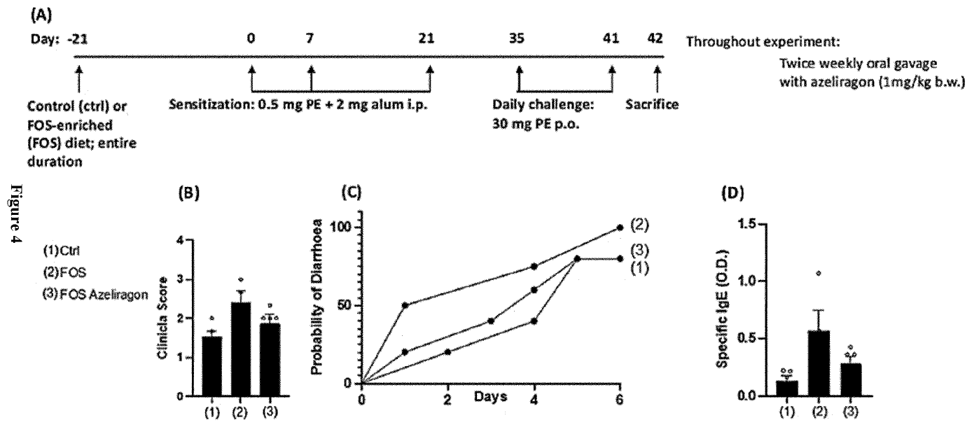

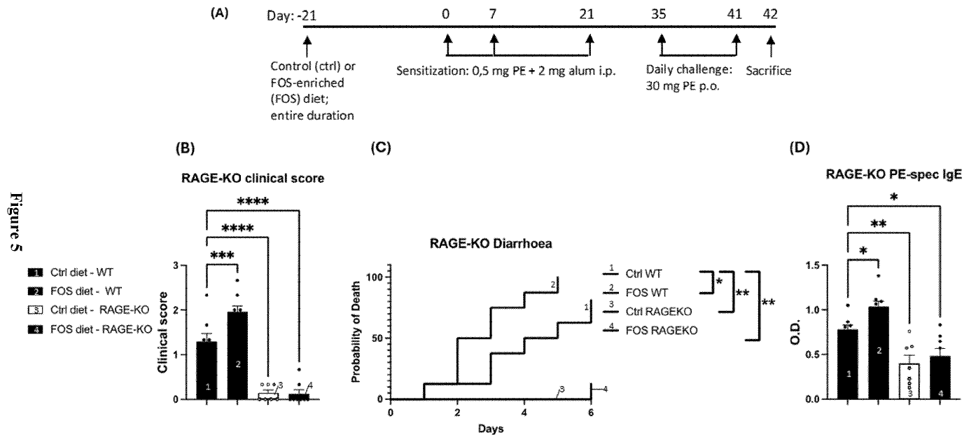

AGEs can activate the receptor for AGEs (RAGE). Our data in FIG. 3 and FIG. 4 demonstrate that two pharmacological antagonists of RAGE, FPS-ZM1 (FIG. 3A, B, C, D), and azeliragon (FIG. 4A, B, C, D), limit the fructo-oligosaccharide (FOS)-induced aggravating effects in a mouse model of peanut allergy, lowering allergen-specific IgE levels, clinical score and incidence of diarrhoea in response to peanut exposure in peanut-sensitized mice. The critical role of RAGE receptor is further underlined by the protection against food allergy offered by a genetic deficiency, FIG. 5 shows that mice that do not express RAGE are protected from food allergy, indicated by the low clinical score (FIG. 1A), absence of diarrhoea after allergen challenge (FIG. 5B), and the lower levels of antigen-specific IgE (FIG. 5C).

### CONCLUSION

Altogether, these data indicate that dietary fructo-oligosaccharide (FOS) can aggravate food allergic reactions through a mechanism involving formation of advanced end glycation products (AGEs) and activation of the RAGE receptor. Therefore, blockade of RAGE represents a promising therapeutic strategy in food allergy.

TABLE 1
Diet composition

| | AIN-93M | AIN93-M-FOS |
|---|---|---|
| Corn-starch | 465.692 | 365.692 |
| Fructo-oligosaccharides | 0 | 100 |
| Casein | 140 | 140 |
| Dextrinized corn-starch | 255 | 155 |
| Sucrose | 0 | 100 |
| Soybean oil | 40 | 40 |
| Powdered cellulose | 50 | 50 |
| Mineral mix (AIN-93M- | 35 | 35 |
| MX) | | |
| Vitamin mix (AIN-93-VX) | 10 | 10 |
| l-Cystein | 1.8 | 1.8 |
| Choline bitartrate | 2.5 | 2.5 |
| Tert-butylhydroquinone | 0.008 | 0.008 |

TABLE 2
Clinical severity score
Clinical severity score

0: no symptoms;
1: scratching and rubbing around the nose and
head;
2: puffiness around the eyes and mouth,
diarrhoea, pilar erecti, reduced activity, and/or
decreased activity with
increased respiratory rate;
3: wheezing, labored respiration, and cyanosis
around the mouth and tail;
intestinal cramping and pain;
4: no activity after prodding or tremor and
convulsion;
5: death.

### REFERENCES

Throughout this application, various references describe the state of the art to which this invention pertains. The disclosures of these references are hereby incorporated by reference into the present disclosure.
Dascalu A E, Furman C, Landrieu I, Cantrelle F X, Mortelecque J, Grolaux G, Gillery P, Tessier F, Lipka E, Billamboz M, Boulanger E, Ghinet A. Development of Receptor for Advanced Glycation End Products (RAGE) ligands through target directed dynamic combinatorial chemistry: a novel class of possible antagonists. Chemistry. 2024 Feb. 6: e202303255.
Folkerts, J., R. Stadhouders, F. A. Redegeld, S. Y. Tam, R. W. Hendriks, S. J. Galli, and M. Maurer. 2018. ‘Effect of Dietary Fiber and Metabolites on Mast Cell Activation and Mast Cell-Associated Diseases’, Front Immunol, 9:1067.
Koh, A., F. De Vadder, P. Kovatcheva-Datchary, and F. Backhed. 2016. ‘From Dietary Fiber to Host Physiology: Short-Chain Fatty Acids as Key Bacterial Metabolites’, Cell, 165:1332-45.
Milutinovic, P. S., J. F. Alcorn, J. M. Englert, L. T. Crum, and T. D. Oury. 2012. ‘The receptor for advanced glycation end products is a central mediator of asthma pathogenesis’, Am J Pathol, 181:1215-25.
Oczypok, E. A., P. S. Milutinovic, J. F. Alcorn, A. Khare, L. T. Crum, M. L. Manni, M. W. Epperly, A. M. Pawluk, A. Ray, and T. D. Oury. 2015. ‘Pulmonary receptor for advanced glycation end-products promotes asthma pathogenesis through IL-33 and accumulation of group 2 innate lymphoid cells’, J Allergy Clin Immunol, 136:747-56 e4.
Pawankar, P. R.; Canonica, G. W.; Holgate, S. T.; Lockey, R. F. 2011. ‘World Health Organization. White Book on Allergy 2011-2012 Executive Summary.’.
Sicherer, S. H., and H. A. Sampson. 2018. ‘Food allergy: A review and update on epidemiology, pathogenesis, diagnosis, prevention, and management’, J Allergy Clin Immunol, 141:41-58.
Staudacher, H. M., and K. Whelan. 2017. ‘The low FODMAP diet: recent advances in understanding its mechanisms and efficacy in IBS’, Gut, 66:1517-27.
Tan, J., C. Mckenzie, P. J. Vuillermin, G. Goverse, C. G. Vinuesa, R. E. Mebius, L. Macia, and C. R. Mackay. 2016. ‘Dietary Fiber and Bacterial SCFA Enhance Oral Tolerance and Protect against Food Allergy through Diverse Cellular Pathways’, Cell Rep, 15:2809-24.
Turner, P. J., and D. E. Campbell. 2016. ‘Epidemiology of severe anaphylaxis: can we use population-based data to understand anaphylaxis?’, Curr Opin Allergy Clin Immunol, 16:441-50.
Ullah, M. A., Z. Loh, W. J. Gan, V. Zhang, H. Yang, J. H. Li, Y. Yamamoto, A. M. Schmidt, C. L. Armour, J. M. Hughes, S. Phipps, and M. B. Sukkar. 2014. ‘Receptor for advanced glycation end products and its ligand high-mobility group box-1 mediate allergic airway sensitization and airway inflammation’, J Allergy Clin Immunol, 134:440-50.
Whelan, K., and H. Staudacher. 2022. ‘Low FODMAP diet in irritable bowel syndrome: a review of recent clinical trials and meta-analyses’, Curr Opin Clin Nutr Metab Care, 25:341-47.
Williams, M. E., W. K. Bolton, R. G. Khalifah, T. P. Degenhardt, R. J. Schotzinger, and J. B. McGill. 2007. ‘Effects of pyridoxamine in combined phase 2 studies of patients with type 1 and type 2 diabetes and overt nephropathy’, Am J Nephrol, 27:605-14.

1. A method of treating a food allergy in a subject in need thereof comprising administering to said subject a therapeutically effective amount of a Receptor for Advanced End Glycation (RAGE) antagonist.

2. The method according to claim 1, wherein the subject suffers from a peanuts allergy.

3. The method according to claim 1, wherein the RAGE antagonist is azeliragon.

4. The method according to claim 1, wherein the RAGE antagonist is FPS-ZM1.

5. The method according to claim 1, wherein the RAGE antagonist is a siRNA.

6. A pharmaceutical composition comprising a RAGE antagonist for use in the treatment of food allergy.

7. The pharmaceutical composition for use according to claim 6 wherein the pharmaceutical composition comprises at least one further therapeutic agent.

8. The pharmaceutical composition according to claim 7 wherein the at least one further therapeutic agent is one or more of an antihistamines, an immunotherapies, a decongestants, a corticosteroids, a steroids, a mast cell stabilizers, a leukotriene modifiers, and adrenalin.

9. A method for preventing food allergy in a subject at risk of being allergic to a food allergen, comprising administering to said subject a therapeutically effective amount of a RAGE antagonist or a pharmaceutical composition comprising a RAGE antagonist.
